(19) United States
(12) Patent Application Publication
(10) Pub. No.: US 2020/0405652 A1
(43) Pub. Date: Dec. 31, 2020

(54) METHYLPHENIDATE-CONTAINING PATCH

(71) Applicant: Hisamitsu Pharmaceutical Co., Inc., Tosu-shi, Saga (JP)

(72) Inventors: Kazuya Kominami, Tsukuba-shi, Ibaraki (JP); Naoyuki Uchida, Tsukuba-shi, Ibaraki (JP); Ryo Tanaka, Tsukuba-shi, Ibaraki (JP); Takito Shima, Tsukuba-shi, Ibaraki (JP)

(73) Assignee: Hisamitsu Pharmaceutical Co., Inc., Tosu-shi, Saga (JP)

(21) Appl. No.: 16/975,807

(22) PCT Filed: Feb. 18, 2019
(86) PCT No.: PCT/JP2019/005817

§ 371 Date: Aug. 26, 2020

(30) Foreign Application Priority Data
Feb. 27, 2018 (JP) 2018-032719

(51) Int. Cl. A61K 9/70; A61K 31/4458

(52) U.S. Cl. CPC A61K 9/7053; A61K 31/4458

(57) ABSTRACT

The present invention addresses the problem of providing a methylphenidate-containing patch that maintains a suitable adhesion to skin over a long period of time and has a sustained drug efficacy and an excellent handeleability. The present invention relates to a patch comprising a backing layer and an adhesive layer, wherein the adhesive layer contains methylphenidate, a rubber-based adhesive base, and a tackifying resin. The rubber-based adhesive base contains a styrene-isoprene-styrene block copolymer, and the tackifying resin contains a terpene-based resin and an alicyclic saturated hydrocarbon resin. The present invention further relates to a method for producing the patch.

### TECHNICAL FIELD

The present invention relates to a patch containing methylphenidate in an adhesive layer and a method for producing the same.

### BACKGROUND ART

Methylphenidate is a central nervous system stimulant used for the treatment of attention deficit/hyperactivity disorder (AD/HD), and its tablet (Ritalin® tablet) or controlled release-type sustained-release tablet (Concerta® tablet) are commercially available. As a dosage form other than oral preparation, Daytrana®, which is a patch, is commercially available overseas.

Several embodiments have been studied as a patch containing methylphenidate; for example, there is description of a patch using a mixture of a silicone adhesive, an acrylic-based adhesive and methylphenidate for the transdermal delivery of methylphenidate to AD/HD patients for at least 10 hours (Patent Document 1).

In addition, regarding the above Daytrana®, there are problems that when a release liner is removed from a polymer matrix containing acrylic polymer, silicone polymer and methylphenidate, the matrix is damaged (Patent Document 2), and that a release liner cannot be peeled off satisfactorily because release force from the release liner increases with time (Patent Document 3); in order to address such problems, the following ideas have been made: various acrylate monomers are mixed in specific ratios (Patent Document 2), and a rubber-based polymer such as styrene-isoprene-styrene block copolymer is used (Patent Document 3).

### CITATION LIST

### Patent Document

[Patent Document 1] JP A No. 2002-510600
[Patent Document 2] WO 2014/062494
[Patent Document 3] WO 2014/159573

### SUMMARY OF INVENTION

### Problems to be Solved by Invention

While proceeding with the study of patches containing methylphenidate, the present inventors have come to recognize a problem that, in conventional patches containing methylphenidate and a rubber-based adhesive base, the adhesion strength to the skin changes with time, and their application for along period of time is difficult; and therefore, sufficient medicinal effect and handleability cannot be expected.

Thus, the object of the present invention is to solve such problems, and to provide a patch containing methylphenidate which maintains an appropriate adhesion strength to the skin for a long period of time, and has a sustained medicinal effect and excellent handleability.

### Means for Solving the Problems

The present inventors have conducted extensive studies to solve such problems, and found that, when the adhesive layer contains methylphenidate and a rubber-based adhesive base, by forming the adhesive layer using a rubber-based adhesive base containing a styrene-isoprene-styrene block copolymer and a tackifying resin containing a terpene-based resin and an alicyclic saturated hydrocarbon resin, a patch that can maintain an appropriate adhesion strength to the skin for a long period of time can be obtained; as a result of further studies, the present inventors have completed the present invention. That is, the present invention relates to the following.

[1] A patch comprising a backing layer and an adhesive layer, wherein
the adhesive layer contains methylphenidate, a rubber-based adhesive base and a tackifying resin,
the rubber-based adhesive base contains a styrene-isoprene-styrene block copolymer, and
the tackifying resin contains a terpene-based resin and an alicyclic saturated hydrocarbon resin.
[2] The patch according to [1], wherein the content of the styrene-isoprene-styrene block copolymer (A) relative to the total amount of the adhesive layer is 20 to 33 mass %.
[3] The patch according to [1] or [2], wherein the content of the terpene-based resin (B) relative to the total amount of the adhesive layer is 10 to 20 mass %.
[4] The patch according to any one of [1] to [3], wherein the content of the alicyclic saturated hydrocarbon resin (C) relative to the total amount of the adhesive layer is 25 to 55 mass %.
[5] The patch according to any one of [1] to [4], wherein, regarding the content of the styrene-isoprene-styrene block copolymer (A), the content of the terpene-based resin (B), and the content of the alicyclic saturated hydrocarbon resin (C) relative to the total amount of the adhesive layer, (A)/(B+C) is in the range of 0.27 to 0.94.
[6] The patch according to any one of [1] to [5], wherein, regarding the content of the styrene-isoprene-styrene block copolymer (A), the content of the terpene-based resin (B), and the content of the alicyclic saturated hydrocarbon resin (C) relative to the total amount of the adhesive layer, (B)/(A+B+C) is in the range of 0.10 to 0.31.
[7] The patch according to any one of [1] to [6], wherein the content of the methylphenidate is 10 to 30 mass %.
[8] A method for producing a patch containing methylphenidate, comprising:
(a) mixing methylphenidate, a styrene-isoprene-styrene block copolymer, a terpene-based resin and an alicyclic saturated hydrocarbon resin to form an adhesive layer,
(b) laminating the adhesive layer and a backing layer.
[9] The production method according to [8], wherein the content of the styrene-isoprene-styrene block copolymer (A) relative to the total amount of the adhesive layer is 20 to 33 mass %.
[10] The production method according to [8] or [9], wherein the content of the terpene-based resin (B) relative to the total amount of the adhesive layer is 10 to 20 mass %.
[11] The production method according to any one of claims 8 to 10, wherein the content of the alicyclic saturated hydrocarbon resin (C) relative to the total amount of the adhesive layer is 25 to 55 mass %.
[12] The production method according to any one of [8] to [11], wherein, regarding the content of the styrene-isoprene-styrene block copolymer (A), the content of the terpene-based resin (B), and the content of the alicyclic saturated hydrocarbon resin (C) relative to the total amount of the adhesive layer, (A)/(B+C) is in the range of 0.27 to 0.94.
[13] The production method according to any one of [8] to [12], wherein, regarding the content of the styrene-isoprene-styrene block copolymer (A), the content of the terpene-based resin (B), and the content of the alicyclic saturated hydrocarbon resin (C) relative to the total amount of the adhesive layer, (B)/(A+B+C) is in the range of 0.10 to 0.31.
[14] The method according to any one of [8] to [13], wherein the content of the methylphenidate is 10 to 30 mass %.
[15] A patch containing methylphenidate produced by the production method according to any one of [8] to [14].

### Advantageous Effects of Invention

According to the present invention, a patch containing methylphenidate can maintain an appropriate adhesion strength to the skin for a long period of time. The adhesion strength to the skin is different from the adhesion strength to the release liner, and the former is extremely important as it has a great impact on sustained medicinal effect of methylphenidate including the effect in the treatment of attention deficit/hyperactivity disorder (AD/HD) while maintaining the form of the patch. In addition, according to the present invention, the stability of methylphenidate in the patch can be maintained for a long period of time. Furthermore, according to the present invention, handling is excellent at the time peeling off the release liner.

### EMBODIMENTS FOR CARRYING OUT INVENTION

The patch of the present invention comprises, for example, a backing layer and an adhesive layer laminated on the backing layer.

The backing may be any one that can maintain the shape of the patch, especially the adhesive layer. Examples of a material of the backing include polyethylene, polypropylene, polybutadiene, ethylene-vinyl chloride copolymer, polyvinyl chloride, polyamide such as Nylon (trade name), polyester, cellulose derivative, synthetic resin such as polyurethane, etc. Nature of the backing is, for example, a film, a sheet, a sheet-like porous body, a sheet-like foam, a textile such as a woven fabric, a knitted fabric and a non-woven fabric, etc., and a laminate thereof. The thickness of the backing is not particularly limited, and usually it is preferably about 2 to 3000 μm.

The adhesive layer contains methylphenidate, a rubber-based adhesive base and a tackifying resin, wherein the rubber-based adhesive contains a styrene-isoprene-styrene block copolymer, and the tackifying resin contains a terpene-based resin and an alicyclic saturated hydrocarbon resin. Furthermore, the patch of the present invention may contain, in addition to methylphenidate, the rubber-based adhesive base and the tackifying resin, if necessary, a plasticizer, an absorption promoter, a stabilizer, a solubilizer, a crosslinking agent, a preservative, a filler, and other additive components such as fragrances.

The methylphenidate of the present invention may be any isomers of methylphenidate including stereoisomers (d-erythro-methylphenidate, 1-erythro-methylphenidate, d-threo-methylphenidate, and 1-threo-methylphenidate), or a derivative or salt thereof; in addition, it is interchangeable with methylphenyl(piperidin-2-yl)acetate, and may be a derivative or salt thereof. The methylphenidate of the present invention may also be a mixture of two or more racemic compounds (such as d/l-erythro-methylphenidate and d/l-threo-methylphenidate).

The content of the above methylphenidate can be appropriately set by those skilled in the art, and based on the total amount of the adhesive layer, it is preferably 10 to 30 mass %, more preferably 15 to 30 mass %, furthermore preferably 18 to 27 mass %, and particularly preferably 20 to 25 mass %.

The rubber-based adhesive base of the present invention contains a styrene-isoprene-styrene block copolymer (hereinafter abbreviated as “SIS”). As the rubber-based adhesive base, SIS may be used alone, or two or more kinds of SISs may be used in combination with other rubber-based adhesive bases. Examples of rubber-based adhesive bases that can be combined with SIS include natural rubber, synthetic rubber, polyisoprene, polyisobutylene, polybutadiene, alkyl vinyl ether (co)polymer, styrene-butadiene-styrene block copolymer, styrene-butadiene rubber, polybutene, styrene-isoprene copolymer, etc.

Specific examples of the rubber-based adhesive base include Oppanol B12, B15, B50, B80, B100, B120, B150, B220 (by BASF, trade name), JSR butyl 065, 268, 365 (by JSR Corporation, trade name), Vistanex LM-MS, MH, H, MML-80, 100, 120, 140 (by Exxon Chemical Company, tradename), HYCAR (by Goodrich Chemical Co., trade name), SIBSTAR T102 (by Kaneka Corporation, trade name) and the like.

In addition, the content of the rubber-based adhesive base can be appropriately set by those skilled in the art in consideration of sufficient adhesion strength and local irritation at the time of peeling of the patch; based on the total amount of the adhesive layer, the content is preferably 10 to 40 mass %, more preferably 15 to 35 mass %, and particularly preferably 20 to 33 mass %. Especially preferably, it is 21 to 29 mass %.

The tackifying resin of the present invention contains a terpene-based resin and an alicyclic saturated hydrocarbon resin. Specific examples of the terpene-based resin include YS resin (by Yasuhara Chemical Co., Ltd., tradename) and Picolite (by Ruth & Dilworth, tradename) and the like. Specific examples of the alicyclic saturated hydrocarbon resin include Alcon® (by Arakawa Chemical Industries, Ltd., trade name), Regalrez (by Eastman Chemical Company, trade name), Piccolastic (by Eastman Chemical Company, trade name), Escoletz (by Exxon Chemical Company, trade name), Wing Tack (by Goodyear Co., trade name), Quintone® (by Zeon Corporation, trade name) and the like.

As the tackifying resin, only two kinds, i.e., terpene-based resin and alicyclic saturated hydrocarbon resin may be used, or three or more kinds may be used by combining other tackifying resins. Examples of the tackifying resin in which two kinds, i.e., terpene-based resin and alicyclic saturated hydrocarbon resin are combined include rosin, rosin derivatives such as rosin glycerin ester, hydrogenated rosin, hydrogenated rosin glycerin ester, and rosin pentaerythritol ester; petroleum resin and maleic acid resin. Specifically, for example, rosin-based resins, phenol-based resins, and xylene-based resins such as ESTER GUM (by Arakawa Chemical Industries, Ltd., trade name), Hariester (by Harima Chemicals Group, Inc., tradename), Pentalyn® (by Eastman Chemical Company, trade name), Foral (by Eastman Chemical Company, trade name) and KE-311 (by Arakawa Chemical Industries, Ltd., trade name), etc., may be mentioned.

The above-mentioned terpene-based resins may be used alone or in combination of two or more. The content of the terpene-based resin can be appropriately set by those skilled in the art in consideration of sufficient adhesion strength and local irritation at the time of peeling of the patch, and based on the total amount of the adhesive layer, it is preferably 1 to 30 mass %, more preferably 5 to 25 mass %, and particularly preferably 10 to 20 mass %.

The above-mentioned alicyclic saturated hydrocarbon resins may be used alone or in combination of two or more. The content of the alicyclic saturated hydrocarbon resin can be appropriately set by those skilled in the art in consideration of sufficient adhesion strength and local irritation at the time of peeling of the patch, and others, and based on the total amount of the adhesive layer, it is preferably 10 to 70 mass %, more preferably 20 to 60 mass %, and particularly preferably 25 to 55 mass %.

The plasticizer may be any of those that impart flexibility to the adhesive layer. Examples of the plasticizer include mineral oils (for example, paraffin oil, naphthenic oil, aromatic oil), animal oils (for example, squalane, squalene), vegetable oils (for example, olive oil, camellia oil, castor oil, tall oil, peanut oil), silicone oils, dibasic acid esters (for example, dibutyl phthalate, dioctyl phthalate), liquid rubbers (for example, liquid polybutene, liquid polyisoprene), liquid fatty acid esters (for example, isopropyl myristate, hexyl laurate, diethyl sebacate, diisopropyl sebacate), polyhydric alcohols (for example, diethylene glycol, polyethylene glycol, propylene glycol, dipropylene glycol), triacetin, triethyl citrate, crotamiton and the like. The plasticizers may be used alone or in combination of two or more.

In addition, the content of the plasticizer can be appropriately set by those skilled in the art in consideration of sufficient plasticity of the patch, and based on the total amount of the adhesive layer, it is preferably 0 to 15 mass %, more preferably 0.5 to 3 mass %, and particularly preferably 1 to 2 mass % (in particular 1 mass %).

The absorption promoter is a component that regulates the skin permeability of methylphenidate or a pharmaceutically acceptable salt thereof. The absorption promoter is not particularly limited as long as it is a compound that has been conventionally recognized to have an absorption promoting action to the skin, and examples thereof include an aliphatic alcohol having 6 to 20 carbon atoms, an aliphatic ether having 6 to 20 carbon atoms, a fatty acid having 6 to 20 carbon atoms, a fatty acid ester having 6 to 20 carbon atoms, a fatty acid amide having 6 to 20 carbon atoms, glycerin, glycerin fatty acid esters, propylene glycols, propylene glycol fatty acid esters, polyethylene glycol and polyethylene glycol fatty acid esters, aromatic organic acid, aromatic alcohol, aromatic organic acid ester, aromatic organic ether (the above compounds may be saturated or unsaturated, may be linear or branched, and may include a cyclic structure), lactic acid esters, acetic acid esters, monoterpene-based compounds, sesquiterpene-based compounds, Azone®, Azone derivatives, sorbitan fatty acid esters (Span®-based), polysorbate-based (Tween®-based), polyoxyethylene hydrogenated castor oils, polyoxyethylene alkyl ethers, sucrose fatty acid esters, and vegetable oils. Specific examples of absorption promoters include caprylic acid, capric acid, caproic acid, lauric acid, myristic acid, palmitic acid, stearic acid, isostearic acid, oleic acid, linoleic acid, linolenic acid, lauryl alcohol, myristyl alcohol, oleyl alcohol, isostearyl alcohol, cetyl alcohol, methyl laurate, hexyl laurate, diethanolamide laurate, isopropylmyristate, myristyl myristate, octyldodecyl myristate, cetyl palmitate, isopropyl palmitate, salicylic acid, methyl salicylate, ethylene glycol salicylate, cinnamic acid, methyl cinnamate, cresol, cetyl lactate, lauryl lactate, ethyl acetate, propyl acetate, geraniol, thymol, eugenol, terpineol, 1-menthol, borneol, d-limonene, isoeugenol, isoborneol, nerol, dl-camphor, glycerin monocaprylate, glycerin monocaprate, glycerin monolaurate, glycerin monooleate, sorbitan monolaurate, sucrose monolaurate, polysorbate 20, propylene glycol, propylene glycol monolaurate, polyethylene glycol monolaurate, polyethylene glycol monostearate, polyoxyethylene lauryl ether, Nikkol® HCO-60 (Nikko Chemicals, Co., Ltd.), Pyrothiodecane®, olive oil, and sorbitan monooleate. The absorption promoters may be used alone or in combination of two or more.

When the adhesive layer contains an absorption promoter, the content of the absorption promoter is preferably 0 to 30 mass %, more preferably 0 to 20 mass % based on the total mass of the adhesive layer.

Examples of the stabilizer include tocopherol and its ester derivatives, ascorbic acid and its ester derivatives, dibutylhydroxytoluene, butylhydroxyanisole, 2-mercaptobenzimidazole and the like. The stabilizers may be used alone or in combination of two or more.

When the adhesive layer contains a stabilizer, the content of the stabilizer is preferably 0 to 5 mass %, and more preferably 0 to 3 mass % based on the total mass of the adhesive layer.

The crosslinking agent is not particularly limited, and preferable examples include amino resins, phenol resins, epoxy resins, alkyd resins, thermosetting resins such as unsaturated polyesters, isocyanate compounds, blocked isocyanate compounds, organic crosslinking agents, in organic crosslinking agents such as metals and metal compounds. Furthermore, the preservative is not particularly limited, and preferred examples include ethyl p-hydroxybenzoate, propyl p-hydroxybenzoate, butyl p-hydroxybenzoate and the like. The filler is not particularly limited, and preferred examples include calcium carbonate, magnesium carbonate, silicates (aluminum silicate, calcium silicate, magnesium silicate, etc.), cellulose derivatives (hydroxypropyl cellulose, hydroxypropylmethyl cellulose, carboxymethyl cellulose, etc.).

When the contents (mass %) of SIS, terpene-based resin and alicyclic saturated hydrocarbon resin relative to the total amount of the adhesive layer of the present invention are A, B and C, respectively, (A)/(B+C) is preferably 0.1 to 1.0, more preferably 0.2 to 0.94, furthermore preferably 0.27 to 0.94, and particularly preferably 0.30 to 0.77. In addition, (B)/(A+B+C) is preferably 0.05 to 0.50, more preferably 0.07 to 0.40, and particularly preferably 0.10 to 0.31.

The patch may further comprise a release liner. The release liner is laminated on the surface of the adhesive layer opposite to the backing. The provision of the release liner tends to reduce attachment of dust or the like to the adhesive layer during storage.

The material of the release liner is not particularly limited, and films generally known to those skilled in the art can be used. Examples of material for the release liner include polyesters such as polyethylene terephthalate and polyethylene naphthalate; polyolefins such as polyethylene and polypropylene; a film of polyvinyl chloride, polyvinylidene chloride, etc.; a laminated film of high-quality paper and polyolefins; a film of Nylon®, aluminum and the like. The material of the release liner is preferably polypropylene or polyethylene terephthalate.

Next, an example of the method for producing the patch of the present invention will be described.

First, a mixture for forming an adhesive layer is prepared. Using a mixer, the above-mentioned methylphenidate, a rubber-based adhesive base, a tackifying resin, and other components are dissolved or dispersed in a solvent of the adhesive base to obtain a mixture for forming an adhesive layer.

As a solvent for the adhesive base, toluene, hexane, ethyl acetate, cyclohexane, heptane, butylacetate, ethanol, methanol, xylene, isopropanol, etc. may be used. These may be appropriately selected depending on the components to be dissolved or dispersed, and one kind may be used alone, or two or more kinds may be mixed and used in combination.

Then, the obtained mixture for forming the adhesive layer is directly spread on a backing to form an adhesive layer, and then a release liner for protecting the adhesive layer is adhered to the adhesive layer; or said mixture is spread on a paper or film that has been subjected to a release treatment to form an adhesive layer, and then placing a backing on it and transfer the adhesive onto the backing by pressure bonding to obtain a patch.

### EXAMPLE

### <Acceleration Test of Adhesion Strength Using Texture Analyzer>

### [Experimental Method]

To the adhesive surface of each patch of Examples 1 to 3 and Comparative Examples 1 to 3 prepared with the compositions shown in Table 1, a stainless steel probe having a diameter of 4 mm was pressed with the condition of 1 N and 1 sec, and then the probe was pulled up with the condition of 1 mm/sec. The value obtained by summing the loads (N-sec) applied from the start of pulling up until the probe was peeled from the adhesive surface was taken as the adhesion strength (N-sec). The change rate of the adhesion strength ΔTack (%) of the patch was obtained by dividing the adhesion strength of the patch stored at 60° C. for 1 week by the adhesion strength of the patch immediately after production, then multiplied by 100. The patch having a desired adhesion strength is defined as those that have a numerical value of the texture analyzer of “90,000 or more” (N·sec) and ΔTack (%) of “100±25%” (hereinafter, referred to as “evaluation criteria”).

TABLE 1
Composition of each patch

| | | | | Comp. | Comp. | Comp. |
|---|---|---|---|---|---|---|
| Component | Ex. 1 | Ex. 2 | Ex. 3 | Ex. 1 | Ex. 2 | Ex. 3 |
| Methylphenidate (mass %) | 20 | 20 | 20 | 20 | 20 | 20 |
| (A) SIS (mass %) | 23 | 27 | 29 | 20 | 39 | 24 |
| (B) Terpene-based | 11 | 14 | 20 | 0 | 8 | 21 |
| resin (mass %) | | | | | | |
| (C) Alicyclic | 45 | 38 | 29 | 59 | 31 | 34 |
| saturated hydrocarbon resin | | | | | | |
| (mass %) | | | | | | |
| Liquid paraffin (mass %) | 1 | 1 | 2 | 1 | 2 | 1 |
| Total (mass %) | 100 | | | | | |
| (A)/(B + C) | 0.41 | 0.52 | 0.59 | 0.34 | 1.00 | 0.44 |
| (B)/(A + B + C) | 0.14 | 0.18 | 0.26 | — | 0.10 | 0.27 |

### [Experimental Result]

Table 2 shows the results of the acceleration test of adhesion strength. It was shown that all the patches of Examples 1 to 3 satisfy the above evaluation criteria, whereas all the patches of Comparative Examples 1 to 3 do not satisfy the above evaluation criteria. In particular, the result of Example 1 (99%) indicates that ΔTack (%) is almost 100%, that is, the adhesion strength of the patch immediately after production is almost completely maintained without any loss when the patch has been stored at 60° C. for 1 week. From these results, it can be understood that by blending the components (A) to (C) shown in Table 1 in the adhesive layer of the patch at specific ratios as shown in the same table, the adhesion strength can be maintained for a long period of time.

TABLE 2
Results of adhesion strength acceleration test

| | | | | Comp. | Comp. | Comp. |
|---|---|---|---|---|---|---|
| | Ex. 1 | Ex. 2 | Ex. 3 | Ex. 1 | Ex. 2 | Ex. 3 |
| Adhesion strength of the | 131027 | 152713 | 120847 | 54170 | 66686 | 141480 |
| patch immediately after | | | | | | |
| production (N · sec) | | | | | | |
| Adhesion strength of the | 130291 | 133347 | 97046 | 81971 | 39104 | 36067 |
| patch after storage at 60° | | | | | | |
| C. for 1 week (N · sec) | | | | | | |
| ΔTack (%) | 99 | 87 | 80 | 151 | 59 | 25 |

1. A patch comprising a backing layer and an adhesive layer, wherein
the adhesive layer contains methylphenidate, a rubber-based adhesive base and a tackifying resin,
the rubber-based adhesive base contains a styrene-isoprene-styrene block copolymer, and
the tackifying resin contains a terpene-based resin and an alicyclic saturated hydrocarbon resin.

2. The patch according to claim 1, wherein the content of the styrene-isoprene-styrene block copolymer (A) relative to the total amount of the adhesive layer is 20 to 33 mass %.

3. The patch according to claim 1, wherein the content of the terpene-based resin (B) relative to the total amount of the adhesive layer is 10 to 20 mass %.

4. The patch according to claim 1, wherein the content of the alicyclic saturated hydrocarbon resin (C) relative to the total amount of the adhesive layer is 25 to 55 mass %.

5. The patch according to claim 1, wherein, regarding the content of the styrene-isoprene-styrene block copolymer (A), the content of the terpene-based resin (B), and the content of the alicyclic saturated hydrocarbon resin (C) relative to the total amount of the adhesive layer, (A)/(B+C) is in the range of 0.27 to 0.94.

6. The patch according to claim 1, wherein, regarding the content of the styrene-isoprene-styrene block copolymer (A), the content of the terpene-based resin (B), and the content of the alicyclic saturated hydrocarbon resin (C) relative to the total amount of the adhesive layer, (B)/(A+B+C) is in the range of 0.10 to 0.31.

7. The patch according to claim 1, wherein the content of the methylphenidate is 10 to 30 mass %.

8. A method for producing a patch containing methylphenidate, comprising:
(a) mixing methylphenidate, a styrene-isoprene-styrene block copolymer, a terpene-based resin and an alicyclic saturated hydrocarbon resin to form an adhesive layer,
(b) laminating the adhesive layer and a backing layer.

9. The production method according to claim 8, wherein the content of the styrene-isoprene-styrene block copolymer (A) relative to the total amount of the adhesive layer is 20 to 33 mass %.

10. The production method according to claim 8, wherein the content of the terpene-based resin (B) relative to the total amount of the adhesive layer is 10 to 20 mass %.

11. The production method according to claim 8, wherein the content of the alicyclic saturated hydrocarbon resin (C) relative to the total amount of the adhesive layer is 25 to 55 mass %.

12. The production method according to claim 8, wherein, regarding the content of the styrene-isoprene-styrene block copolymer (A), the content of the terpene-based resin (B), and the content of the alicyclic saturated hydrocarbon resin (C) relative to the total amount of the adhesive layer, (A)/(B+C) is in the range of 0.27 to 0.94.

13. The production method according to claim 8, wherein, regarding the content of the styrene-isoprene-styrene block copolymer (A), the content of the terpene-based resin (B), and the content of the alicyclic saturated hydrocarbon resin (C) relative to the total amount of the adhesive layer, (B)/(A+B+C) is in the range of 0.10 to 0.31.

14. The production method according to claim 8, wherein the content of the methylphenidate is 10 to 30 mass %.

15. A patch containing methylphenidate produced by the production method according to claim 8.
